(19) United States
(12) Patent Application Publication
(10) Pub. No.: US 2023/0329625 A1
(43) Pub. Date: Oct. 19, 2023

(54) BIO-SIGNAL MEASUREMENT BAND

(71) Applicant: HHS CO., LTD., Ulsan (KR)

(72) Inventors: Hyung-Seob Han, Ulsan (KR); Yong-Seog Han, Ulsan (KR)

(21) Appl. No.: 18/044,197

(22) PCT Filed: Nov. 27, 2020
(86) PCT No.: PCT/KR2020/017028

§ 371 Date: Mar. 6, 2023

(30) Foreign Application Priority Data
Sep. 8, 2020 (KR) 10-2020-0114499

(51) Int. Cl. A61B 5/369; A61B 5/00

(52) U.S. Cl. CPC A61B 5/369; A61B 5/7405; A61B 5/746; A61B 2562/182

(57) ABSTRACT

The present invention provides a technology relating to a bio-signal measurement band which may prevent intrusion of foreign matter from the outside and reduce interference from external electromagnetic waves. Since intrusion of foreign matter from the outside is prevented, there is an advantage in that damage and deformation occurring within the bio-signal measurement band is prevented and thus the accuracy of measured bio-signals may be improved. In addition, since the bio-signal measurement band has a structure in which the bio-signal measurement band is shielded except the electrode thereof, there is an advantage in that the problem of a reduction of accuracy due to external electromagnetic waves is solved and thus bio-signals may be measured with improved accuracy.

### CROSS-REFERENCE TO RELATED APPLICATION(S)

This application is a Section 371 National Stage Application of International Application No. PCT/KR2020/017028, filed on Nov. 27, 2020, which claims priority to Republic of Korea Application No. 10-2020-0114499, filed Sep. 8, 2020, the whole disclosures of which are incorporated herein by reference in their entirety.

### BACKGROUND

### Technical Field

The present disclosure relates to a bio-signal measurement band that is placed against a user's forehead to measure a plurality of bio-signals including brain waves, and more specifically, to the bio-signal measurement band preventing inflow of foreign substances from an outside and reducing interference with external electromagnetic waves.

### Description of Related Art

A device installed on a portion of the user's head to measure the user's brain wave means a device for measuring a brain wave signal through an electrode attached to the head and preprocessing the measured brain wave signal and extracting feature elements therefrom.

The brain measurement technique may be classified into invasive and non-invasive types. In the invasive type, a microchip is implanted into a scalp to measure brain waves. Thus, the invasive type can measure the brain wave accurately, but requires a procedure and has surgical side effects. In a representative example thereof, Duke University's research results have been published in which a brain wave measurement microchip is implanted in the brain of a monkey and an arm of a machine is moved using a nerve signal detected in the brain.

It is easier to measure brain waves in the non-invasive type than in the invasive type. The non-invasive type is convenient in that the brain wave is measured with a helmet or headset type equipment. However, noise signals are inevitably mixed with the brain wave in the non-invasive type. Therefore, there is a problem in the non-invasive type in that it is difficult to accurately measure the brain wave. The non-invasive type device is used in labs of universities or research institutes and some commercialized products thereof are open to the public. However, the accuracy of the non-invasive type device is relatively low due to the noise signals. Thus, it is difficult to use the non-invasive type device in practice.

### SUMMARY

The present disclosure is intended to solve the problems of the prior art, and the purpose of the present disclosure is to provide a bio-signal measurement band capable of preventing the inflow of foreign substances from the outside and reducing interference from external electromagnetic waves.

Purposes in accordance with the present disclosure are not limited to the above-mentioned purpose. Other purposes and advantages in accordance with the present disclosure as not mentioned above may be appreciated from following descriptions and more clearly appreciated from embodiments in accordance with the present disclosure. Further, it will be readily appreciated that the purposes and advantages in accordance with the present disclosure may be realized by features and combinations thereof as disclosed in the claims.

A bio-signal measurement band includes at least one electrode for measuring at least one bio-signal including a brain wave; a housing composed of a first housing and a second housing made of an electromagnetic wave shielding material and coupled to each other to define an inner space therebetween, wherein an electrical wire, a circuit, and a battery connected to the electrode are received in the inner space; a sealing extending along a circumference of the electrode and in an annular manner, wherein a portion of the electrode is inserted into the sealing through an inner side face thereof, wherein the first housing is inserted into the sealing through an outer side face thereof, wherein the sealing connects and fixes the electrode and the first housing to each other, wherein the sealing seals the inner space; and a silicon pad covering a side of the electrode, the first housing and the sealing so as to face toward a skin of a user, wherein the electrode and the silicon pad contact the skin of the user.

In one embodiment, the electrode includes: a skin contact portion in contact with the skin of the user; and a non-contact portion inserted into the sealing through the inner side face thereof, wherein a diameter of the skin contact portion is smaller than a diameter of the non-contact portion, wherein the skin contact portion protrudes from the non-contact portion.

In one embodiment, the sealing includes: a pair of electrode contact portions defining the inner side face of the sealing; and a pair of housing contact portions defining the outer side face of the sealing, wherein the pair of electrode contact portions contact the non-contact portion of the electrode, wherein the pair of housing contact portions contact the first housing.

In one embodiment, the silicon pad includes an upper contact portion protruding toward the skin of the user beyond the skin contact portion of the electrode, wherein the upper contact portion prevents sweat flowing from a scalp of the user from flowing toward the electrode.

In one embodiment, the silicon pad further includes an annular protrusion extending along a circumference of the skin contact portion of the electrode, wherein the annular protrusion has the same protruding height as a height by which the skin contact portion of the electrode protrudes toward the skin of the user, wherein the annular protrusion prevents sweat flowing from a scalp and a forehead of the user from contacting the electrode.

According to the present disclosure, the inflow of foreign substances from the outside into the band may be prevented, thereby preventing damage and deformation of the bio-signal measurement band and thus improving the accuracy of the measured bio-signal.

Further, according to the present disclosure, the wire, the circuit, the battery connected to the electrode except for a portion of the electrode are shielded from the external electromagnetic wave. Thus, the reduction of the measurement accuracy due to the external electromagnetic wave may be suppressed. Thus, the accuracy of the bio-signal as measured is improved.

In addition to the effects as described above, specific effects in accordance with the present disclosure will be described together with following detailed descriptions for carrying out the disclosure.

### BRIEF DESCRIPTION OF DRAWINGS

FIG. 1 is a perspective view of a bio-signal measurement band according to the present disclosure.

FIG. 2 and FIG. 3 are perspective views of an electrode according to the present disclosure.

FIG. 4 is a cross-sectional perspective view of a sealing according to the present disclosure.

FIG. 5 is a cross-sectional view of a state in which an electrode and a housing are inserted into a sealing according to the present disclosure.

FIG. 6 and FIG. 7 are side cross-sectional views of a bio-signal measurement band according to the present disclosure.

### DETAILED DESCRIPTION

The above objects, features and advantages will be described in detail later with reference to the accompanying drawings. Accordingly, a person with ordinary knowledge in the technical field to which the present disclosure belongs will be able to easily implement the technical idea of the present disclosure. In describing the present disclosure, when it is determined that a detailed description of a known component related to the present disclosure may unnecessarily obscure gist the present disclosure, the detailed description is omitted. Hereinafter, a preferred embodiment according to the present disclosure will be described in detail with reference to the accompanying drawings.

Prior to describing a preferred embodiment of the present disclosure, it is mentioned that a bio-signal measurement band 1000 according to the present disclosure is placed on the user's forehead to measure a bio-signal including brain waves. When the band only contacts the user's forehead, the band is not fixed thereto. Thus, a separate mounting part is required. For example, a preferred embodiment of the present disclosure is fixed to a head holder as a mounting part located inside a helmet. Therefore, a strength by which the band is pressed toward the head (forehead) is controlled by adjusting the head holder as the mounting part.

However, the purpose of the bio-signal measurement band 1000 according to the present disclosure is to prevent foreign substances from entering the inside thereof and to reduce interference with the external electromagnetic wave. Thus, a scheme and a configuration for mounting the band to the head (forehead) is not limited particularly. That is, the bio-signal measurement band 1000 according to the present disclosure may be fixed to the head using a headband, or may be fixed to a general hat or may be mounted on the head holder of the safety helmet in a preferred embodiment of the present disclosure.

Further, the bio-signal measurement band 1000 according to the present disclosure should include a battery, a circuit, a charging port, etc. However, the purpose of the bio-signal measurement band 1000 according to the present disclosure is to prevent foreign substances from entering the inside thereof and to reduce interference with the external electromagnetic wave. Thus, the location of each of the battery, the circuit, the charging port, etc. is not limited particularly and does not limit the scope of the present disclosure.

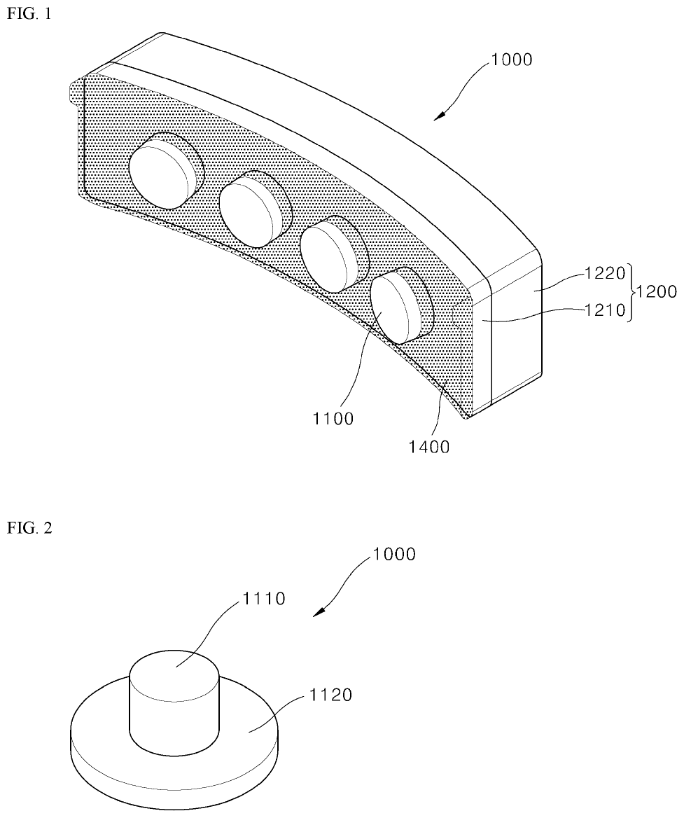

FIG. 1 is a perspective view of the bio-signal measurement band 1000 according to the present disclosure.

Referring to FIG. 1, the bio-signal measurement band 1000 according to the present disclosure includes an electrode 1100 measuring at least one of a brain wave or a bio-signal of the user, a housing 1200 acting as a shielding material and composed of a first housing 1210 and a second housing 1220 coupled to each other, wherein the housing 1200 receives therein and protects a wire, a circuit, and a battery connected to the electrode 1100, a sealing 1300 that connects and fixes the electrode 1100 and the first housing 1210 to each other and seals the inner space of the housing, and a silicon pad 1400 covering one side of the electrode 1100, the first housing 1210, and the sealing 1300 so as to face toward the user's skin, wherein the silicon pad 1400 together with the electrode 1100 comes into contact with the user's skin.

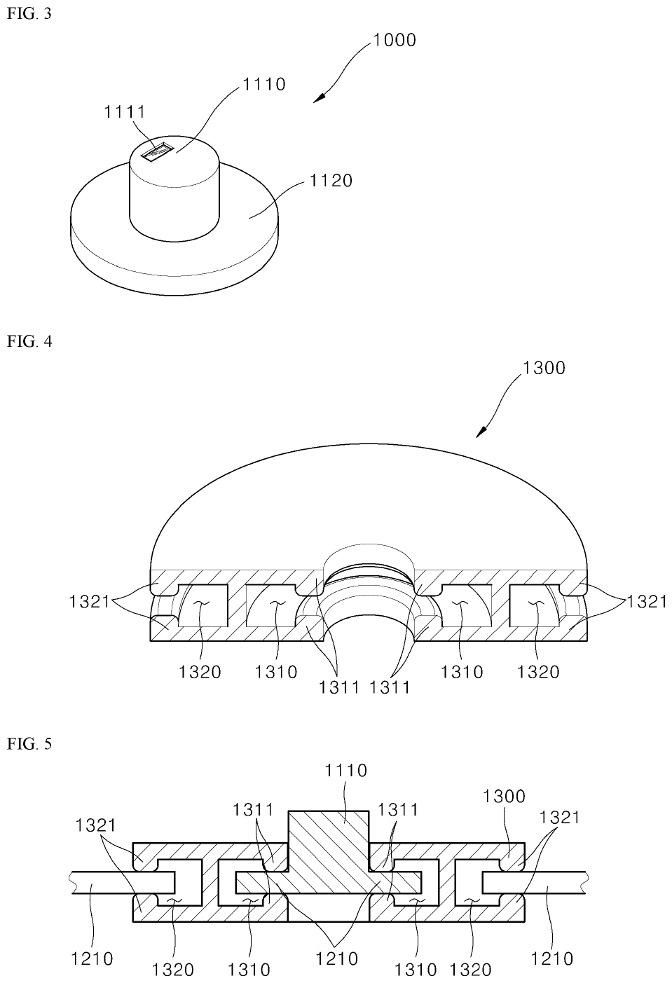

FIG. 2 and FIG. 3 are perspective views of the electrode 1100 according to the present disclosure.

Referring to FIG. 2 and FIG. 3, the electrode 1100 measures at least one of the brain wave or the bio-signal. This means that the electrode 1100 may measure only the brain wave, measure only the bio-signal, or measure the brain wave and the bio-signal at the same time. Further, the electrode 1100 is made of a conductive material. In the present disclosure, an example in which the electrode 1100 is a dry electrode, and is embodied as a gold-plated electrode 1100 is disclosed. However, the electrode 1100 may include silver, copper, aluminum, conductive silicon, nano materials, etc.

The electrode 1100 has a convex front end, and is composed of a skin contact portion 1110 that contacts the user's skin and a non-contact portion 1120 that is inserted into the sealing 1300 to be described later. In this regard, a cross-sectional area of the skin contact portion 1110 is smaller than that of the non-contact portion 1120. That is, the skin contact portion 1110 has a cross-sectional area smaller than that of the non-contact portion 1120 and has a protruding shape extending from the non-contact portion 1120. Therefore, in a state where the non-contact portion 1120 is fixed to the sealing 1300 to be described later, the skin contact portion 1110 contacts the user's skin to measure at least one of the brain wave or the bio-signal.

The skin contact portion 1110 comes into contact with the user's forehead to measure at least one of the brain wave or the bio-signal of the user. To this end, the skin contact portion 1110 includes a gold-plated electrode. However, as described above, the electrode is also not limited to the gold-plated electrode, and may include any material for carrying out the present disclosure.

The non-contact portion 1120 is inserted into the sealing 1300 to be described later to fix the electrode 1100 thereto. To this end, the non-contact portion 1120 may be integral with the skin contact portion 1110 and may be made of the same material as that thereof. However, when the skin contact portion 1110 and the non-contact portion 1120 are made of the same material, the accuracy of the measured brain wave and/or bio-signal may be reduced due to interference of the non-contact portion 1120 with the external electromagnetic wave. Therefore, in the preferred embodiment of the electrode 1100 of the present disclosure, the skin contact portion 1110 and the non-contact portion 1120 are integrally formed with each other but are made of different materials. The skin contact portion 1110 may be preferably made of a conductive material, and the non-contact portion 1120 may be made of a non-conductive material. More preferably, the skin contact portion 1110 may be made of a conductive material, and the non-contact portion may be made of a non-conductive and shielding material.

In this regard, the skin contact portion 1110 of the electrode 1100 according to the present disclosure may have a cut portion 1111 in a surface thereof in contact with the user's skin. The cut portion 1111 measures a bio-signal corresponding to a signal that cannot be measured with a conductive electrode. Therefore, the bio-signal may be measured simultaneously with the measurement of the brain wave.

The bio-signal to be measured in the present disclosure may include electrocardiogram (ECG/EKG), electromyography (EMG), pulse wave/pulsation, photoplethysmogram (PPG), electrical skin reflex (GSR), heart rate variability (HRV), oxygen saturation (SPO₂), pulse transit time (PPT), functional Near-Infrared Spectroscopy (fNIRS), electrooculography (EOG), blood pressure, and body temperature. However, this is a typical bio-signal measurable in the bio-signal measurement band according to the present disclosure, and the type of the bio-signal is not limited thereto.

The housing 1200 includes the first housing 1210 connected to the sealing 1300 connected to and fixed to the non-contact portion 1120 of the electrode 1100, and the second housing 1220 connected and fixed to the first housing 1210 to seal an inside of the housing. That is, the housing 1200 is composed of the first housing 1210 and the second housing 1220 combined to each other so as to define an inner space to accommodate therein and protect the wire, the circuit, and the battery connected to the electrode 1100.

In this regard, means and a scheme of coupling the first housing 1210 and the second housing 1220 to each other are not limited to specific means and scheme. That is, the means and the scheme of coupling the first housing 1210 and the second housing 1220 to each other are optional embodiments for implementing the present disclosure, and are not limited particularly.

Further, the housing 1200 is implemented via the combination of the first housing 1210 acting as a shielding material, and the second housing 1220 acting as a shielding material. Thus, the interference with the electromagnetic wave may be suppressed in all directions except for a position of each of the electrode 1100 and the sealing 1300. Accordingly, the interference of the circuit and the wire with the electromagnetic wave may be prevented, and thus, accuracy of the bio-signal including the brain wave as measured is improved.

In this regard, an outer circumferential surface of a portion at which the first housing 1210 and the second housing 1220 are coupled to each other is concave inwardly. Accordingly, a sealing ring 1500 is inserted into and extends along the concave outer circumferential surface at a position where the first housing 1210 and the second housing 1220 come into contact with each other. The sealing ring 1500 extends along the concave outer circumferential surface and does not protrude beyond an outer circumferential surface of each of the first housing 1210 and the second housing 1220. This means that a thickness of the sealing ring is not larger than a depth of the concave portion defined in the outer circumferential surface where the first housing 1210 and the second housing 1220 come into contact with each other.

In this regard, the depth of the concave portion defined in the outer circumferential surface where the first housing 1210 and the second housing 1220 come into contact with each other is not uniform. More specifically, a portion where the first housing 1210 and the second housing 1220 come into contact with each other may be defined into four portions: upper, lower, and both opposing side portions. In this regard, when the bio-signal measurement band 1000 according to the present disclosure comes into contact with the head (forehead) of the user, the depth of the concave portion defined in the outer circumferential surface where the first housing 1210 and the second housing 1220 come into contact with each other may be sized such that the upper portion is deeper than each of the lower and both opposing side portions. Thus, the depth of the concave portion defined in the outer circumferential surface where the first housing 1210 and the second housing 1220 come into contact with each other may be sized such that the upper portion is deeper than each of the lower and both opposing side portions, such that when the sealing ring 1500 is inserted into the concave portion, the upper portion is not fully filled with the sealing ring. Thus, the user's sweat may flow along an empty space of the upper portion toward both opposing side portions. Therefore, the empty space together with an upper contact portion 1410 of the silicon pad 1400 may prevent the user's sweat from flowing toward the electrode 1100. Thus, the sweat does not flow to the surface of the electrode 1000. Thus, the reduction of the measurement accuracy of the bio-signal due to the sweat may be prevented. Further, the sealing ring may prevent foreign substances such as the sweat from being introduced into the bio-signal measurement band 1000. Therefore, the housing 1200 acting as a shielding material not only may prevent the external electromagnetic wave interference, but also may prevent the foreign substances such as the sweat from being introduced into the bio-signal measurement band 1000 and thus prevent the band 1000 from being damaged due to the foreign substances.

FIG. 4 is a cross-sectional perspective view of the sealing 1300 according to the present disclosure, and FIG. 5 is a cross-sectional view of a state in which the electrode 1100 and the housing 1200 are inserted into the sealing 1300 according to the present disclosure.

Referring to FIG. 4 and FIG. 5, the sealing 1300 extends along the circumference of the electrode 1100 and in an annular manner. More specifically, the sealing 1300 extends along the circumference of the non-contact portion 1120 of the electrode 1100 and in an annular manner. Thus, the non-contact portion 1120 is inserted into an inner space defined by an inner side face of the sealing 1300, such that the sealing 1300 is connected to and fix the non-contact portion 1120 of the electrode 1100. Further, the inner side face of the sealing 1300 is connected to and fixes the non-contact portion 1120 of the electrode 1100, while an outer side face of the sealing 1300 is connected to and fix the first housing 1210. Therefore, the sealing 1300 is connected to and fix each of the non-contact portion 1120 of the electrode 1100 and the first housing 1210 of the housing 1200 thereto.

The sealing 1300 has the inner side face and the outer side face depressed inwardly, and having an opening defined therein. Therefore, the sealing 1300 has an inner opening 1310 formed to be open inwardly and an outer opening 1320 formed to face outwardly.

The inner opening 1310 is defined by a pair of electrode contact portions 1311 facing each other and spaced from each other and defining the inner circumferential surface of the sealing 1300. The pair of electrode contact portions 1311 defines an inner side surface of the inner opening 1310. Accordingly, the pair of electrode contact portions 1311 come into contact with the non-contact portion 1120 of the electrode 1100.

In this regard, the pair of electrode contact portions 1311 defines the inner side surface of the inner opening 1310. The non-contact portion 1120 of the electrode 1100 extends outwardly of the pair of electrode contact portion 1311. Therefore, an edge portion of the skin contact portion 1110 of the electrode 1100 is positioned inwardly of the pair of electrode contact portions 1311, and is received in the inner opening 1310 and thus the skin contact portion 1110 of the electrode 1100 can move up and down in the inner opening 1310. However, when the skin contact portion 1110 of the electrode 1100 moves up and down in the inner opening 1310, foreign substances are not introduced inwardly of the pair of electrode contact portions 1311 due to the pressurization of the pair of electrode contact portions 1311. Therefore, the inside of the electrode 1100 may be protected from the foreign substances introduced from the outside, and the wire, the circuit, battery, etc. connected to the electrode 1100 located inside the housing 1200 may be prevented from being damaged. Thus, the reduction in the measurement accuracy may be suppressed.

The outer opening 1320 is defined by a pair of housing contact portions 1321 facing each other and spaced from each other and defining the outer circumferential surface of the sealing 1300. The pair of housing contact portions 1321 define the outer side surface of the outer opening 1320. Accordingly, the pair of housing contact portions 1321 come into contact with the first housing 1210 of the housing 1200.

In this regard, the pair of housing contact portions 1321 define the outer side surface of the outer opening 1320. Thus, the first housing 1210 of the housing 1200 extends inwardly of the pair of housing contact portions 1321 and is received in the outer opening 1320. Therefore, the first housing 1210 extends inwardly of the pair of housing contact portions 1321 and is received in the outer opening 1320 and can move up and down in the outer opening 1320. However, when the first housing 1210 moves up and down in the outer opening 1320, foreign substances are not introduced inwardly of the pair of housing contact portions 1321 due to the pressurization of the housing contact portions 1321. Therefore, the inside of the electrode 1100 may be protected from the foreign substances introduced from the outside, and the wire, the circuit, battery, etc. connected to the electrode 1100 located inside the housing 1200 may be prevented from being damaged. Thus, the reduction in the measurement accuracy may be suppressed.

In this regard, the sealing 1300 may be made of a rubber material generally used for sealing. Further, the sealing 1300 extends in an annular manner. Thus, the non-contact portion 1120 of the electrode 1100 is circular, the sealing 1300 has the inner opening 1310 having a circular inner circumferential surface. Further, when the non-contact portion of the electrode 1100 has a square shape, the sealing 1300 has the inner opening 1310 having a square shaped inner circumferential surface.

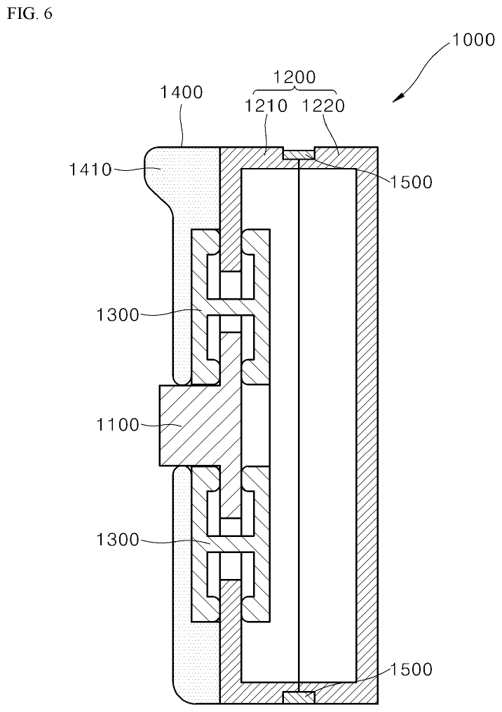

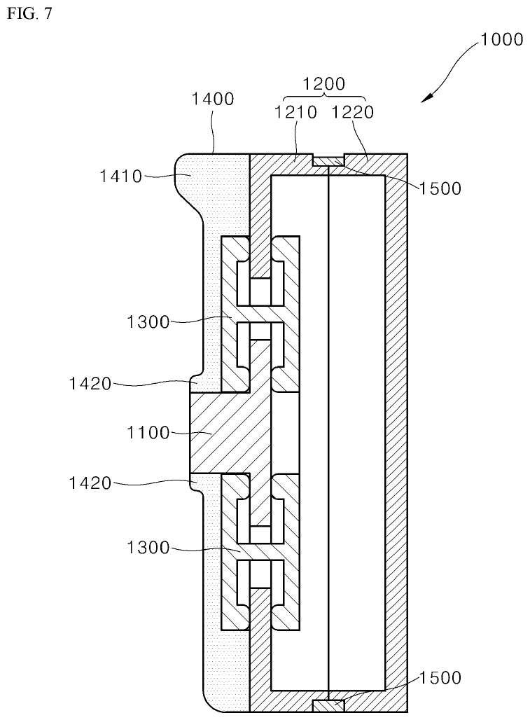

FIG. 6 and FIG. 7 are side cross-sectional views of the bio-signal measurement band 1000 according to the present disclosure.

Referring to FIG. 6 and FIG. 7, the silicon pad 1400 is formed to cover the side surface of the skin contact portion 1110, the first housing 1210, and an area of the sealing 1000 facing the user's skin.

In this regard, in a cross section of the silicon pad 1400, the silicon pad 1400 includes an upper contact portion 1410 which protrudes towards the user's skin beyond the protruding contact portion 1110 of the electrode 1100. Therefore, the upper contact portion 1410 of the silicon pad 1400 is closely adhered to the forehead of the user. Thus, the sweat flowing down from a scalp and the forehead may be prevented from flowing toward the electrode 1100. Therefore, the sweat may be prevented from contacting the electrode 1100 and thus the electrode may be prevented from being corroded. The reduction of the measurement accuracy of the brain wave and/or bio-signal due to the sweat contacting the electrode 1100 may be prevented.

Further, the silicon pad 1400 includes an annular protrusion 1420 extending along the circumference of the protruding contact portion 1110 of the electrode 1100. The annular protrusion 1420 has the same protruding height as a height by which the skin contact portion 1110 protrudes toward the user's skin. Therefore, the skin contact portion 1110 of the electrode 1100 and the annular protrusion 1420 contact the skin at the same pressure applied to the skin. Thus, a combination of the annular protrusion 1420 and the upper contact portion 1410 may prevent the sweat from flowing toward the skin contact portion 1110.

In this regard, the bio-signal measurement band 1000 according to the present disclosure comes into contact with the user's forehead, and thus, it is preferable that the bio-signal measurement band 1000 has a gently curved structure. However, in this case, the electrode 1100 may not easily contact the forehead because an angle at which the electrode 1100 comes into contact with the user's forehead varies based on the user. However, the electrode 1100 of the present disclosure may be movable in the opening defined in the sealing 1300. Thus, due to the pressure applied to the user's forehead, the electrode 1100 partially moves such that the band fits the user's forehead contact surface.

Further, according to another embodiment of the present disclosure, the bio-signal measurement band 1000 includes the electrode 1100 measuring at least one of a brain wave or a bio-signal of the user, the housing 1200 acting as a shielding material and composed of the first housing 1210 and the second housing 1220 coupled to each other, wherein the housing 1200 receives therein and protects a wire, a circuit, and a battery connected to the electrode 1100, the sealing 1300 that connects and fixes the electrode 1100 and the first housing 1210 to each other and seals the inner space of the housing, and the silicon pad 1400 covering one side of the electrode 1100, the first housing 1210, and the sealing 1300 so as to face toward the user's skin, wherein the silicon pad 1400 together with the electrode 1100 comes into contact with the user's skin. Further, the band 1000 further includes an event sensor that determines whether an emergency occurs based on the user's location and acceleration, and a working environment sensor that determines whether or not the user can work based on the air quality around the user.

In this regard, each of the electrode 1100, the housing 1200, the sealing 1300, and the silicon pad 1400 has the same configuration as each of those of the above embodiment. Thus, the description thereof is omitted.

The event sensor measures the user's position and acceleration. The event sensor detects and determines whether or not there is a sudden change in the user's state, such as the user's fall down or collapse. Further, the event sensor determines whether the emergency situation to the user has occurred, based on the brain wave and/or bio-signal measured by the electrode 1100.

The working environment sensor measures the presence or absence of harmful gases in the air around the user, the concentrations of oxygen and harmful gases, the outside temperature, and a fine dust amount.

The working environment sensor may be formed on the outer side face of the aforementioned housing or is a separate component extending from the housing. Therefore, the bio-signal measurement band 1000 according to the present disclosure measures the presence or absence of harmful gases around the user, the concentrations of oxygen and harmful gases, the external temperature, and the fine dust amount, and determines whether the user can work based on the measurements, and warns the user of the harmful gas. To this end, a harmful gas meter further includes separate warning means.

Desirable first warning means is sound. In this case, a speaker is provided to give a warning to the user to stop or prohibit the work because a value of the harmful gas of the ambient air around the user currently exceeds a reference value.

When the user does not stop working or enters an area in which the value of the harmful gas is higher than the reference value despite the sound based warning, it is determined that the first sound based warning is not recognized by the user, and a second warning is performed.

In this regard, second warning means is to temporarily apply a strong current to the skin contact portion 1110 of the electrode 1100 to apply a fine electric stimulus to the user. Accordingly, when the user does not recognize the first sound based warning, the user may recognize a dangerous situation via the electrical stimulation.

Therefore, according to the present disclosure, the foreign substances may be prevented from invading the band. The interference with the electromagnetic wave may be reduced due to the housing 1200 made of the shielding material. Moreover, the status of the user and the work environment may be determined to cope with the emergency situation in an earlier manner.

The present disclosure has been described above with reference to exemplified drawings. However, the present disclosure is not limited to the embodiments and drawings as disclosed in the present disclosure. It is obvious that the person skilled in the art may make various modifications within the scope of the technical idea of the present disclosure. In addition, although the effect according to the configuration of the present disclosure is not explicitly described while describing the embodiment of the present disclosure, it is natural that the effect predictable from the configuration should also be recognized.

1. A bio-signal measurement band comprising:
at least one electrode for measuring at least one bio-signal including a brain wave;
a housing composed of a first housing and a second housing made of an electromagnetic wave shielding material and coupled to each other to define an inner space therebetween, wherein an electrical wire, a circuit, and a battery connected to the electrode are received in the inner space;
a sealing extending along a circumference of the electrode and in an annular manner, wherein a portion of the electrode is inserted into the sealing through an inner side face of the sealing, wherein the first housing is inserted into the sealing through an outer side face of the sealing, wherein the sealing connects and fixes the electrode and the first housing to each other, wherein the sealing seals the inner space; and
a silicon pad covering a side of the electrode, the first housing and the sealing so as to face toward a skin of a user, wherein the electrode and the silicon pad are configured to contact the skin of the user.

2. The bio-signal measurement band of claim 1, wherein the electrode includes:
a skin contact portion configured to contact with the skin of the user; and
a non-contact portion inserted into the sealing through the inner side face thereof,
wherein a diameter of the skin contact portion is smaller than a diameter of the non-contact portion,
wherein the skin contact portion protrudes from the non-contact portion.

3. The bio-signal measurement band of claim 2, wherein the sealing includes:
a pair of electrode contact portions defining the inner side face of the sealing; and
a pair of housing contact portions defining the outer side face of the sealing,
wherein the pair of electrode contact portions contact the non-contact portion of the electrode,
wherein the pair of housing contact portions contact the first housing.

4. The bio-signal measurement band of claim 3, wherein the silicon pad includes an upper contact portion configured to protrude toward the skin of the user beyond the skin contact portion of the electrode,
wherein the upper contact portion is configured to prevent sweat flowing from a scalp of the user from flowing toward the electrode.

5. The bio-signal measurement band of claim 4, wherein the silicon pad further includes an annular protrusion extending along a circumference of the skin contact portion of the electrode, wherein the annular protrusion has the same protruding height as a height by which the skin contact portion of the electrode protrudes toward the skin of the user,
wherein the annular protrusion is configured to prevent sweat flowing from a scalp and a forehead of the user from contacting the electrode.

6. A bio-signal measurement band comprising:
at least one electrode for measuring at least one bio-signal including a brain wave;
a housing composed of a first housing and a second housing made of an electromagnetic wave shielding material and coupled to each other to define an inner space therebetween, wherein an electrical wire, a circuit, and a battery connected to the electrode are received in the inner space; and
a sealing extending along a circumference of the electrode and in an annular manner, wherein a portion of the electrode is inserted into the sealing through an inner side face of the sealing, wherein the first housing is inserted into the sealing through an outer side face of the sealing, wherein the sealing connects and fixes the electrode and the first housing to each other, and wherein the sealing seals the inner space,
wherein a recess is defined in an outer circumferential surface of an area in which the first housing and the second housing are coupled to each other,
wherein a sealing ring is inserted into and extends along the recess,
wherein the recess extends along and is defined in top and bottom and side portions of the housing,
wherein a depth of the recess is sized such that a depth of the recess defined in the top portion is larger than a depth of the recess defined in each of the bottom and side portions,
wherein a thickness of the sealing ring is smaller than the depth of the recess.

7. A bio-signal measurement band comprising:
at least one electrode for measuring at least one bio-signal including a brain wave;
a housing composed of a first housing and a second housing made of an electromagnetic wave shielding material and coupled to each other to define an inner space therebetween, wherein an electrical wire, a circuit, and a battery connected to the electrode are received in the inner space;
an event sensor configured to measure an acceleration and a position of a user; and
a working environment sensor configured for measuring at least one of presence or absence of a harmful gas, a level of each of oxygen and the harmful gas, an outside temperature, or a fine dust amount,
wherein the working environment sensor determines whether it is possible for the user to work in the working environment,
wherein upon determination that it is not possible for the user to work in the working environment, the working environment sensor performs a first warning based on a sound,
wherein when the first warning is not effective, the working environment sensor performs a second warning, wherein the second warning includes applying electrical current to the electrode to stimulate the user.
